(19) United States
(12) Patent Application Publication
(10) Pub. No.: US 2025/0152947 A1
(43) Pub. Date: May 15, 2025

(54) Craniometry Implant Tray Having A Ceramic Insert For Improved Remote Charging And Two-Way Communication

(71) Applicant: Greatbatch Ltd., Clarence, NY (US)

(72) Inventors: Agustin Villavedra, Montevideo (UY); Ignacio Agustin Armesto, Montevideo (UY); Luis Daniel Villamil, Montevideo (UY)

(21) Appl. No.: 18/939,668

(22) Filed: Nov. 7, 2024

Related U.S. Application Data
(60) Provisional application No. 63/547,993, filed on Nov. 10, 2023.

(51) Int. Cl. A61N 1/372; A61N 1/375

(52) U.S. Cl. CPC A61N 1/37282; A61N 1/3758

(57) ABSTRACT

A medical tray that is useful for containing an implantable medical device is a body cavity is described. The medical tray is particularly useful in a craniometry and comprises a ceramic insert joined to a metallic main tray. The main tray comprises a base plate extending to an upstanding main sidewall. An inlet extends inwardly through the main sidewall and into the base plate with the inlet having an inlet peripheral edge. The ceramic insert comprises a planar plate portion extending to an upstanding insert sidewall. The insert has an insert peripheral edge having an insert shape that is conformal with an inlet shape of the inlet peripheral edge. A metallization is supported on the insert peripheral edge and a braze joins the metallization supported on the insert peripheral edge to the inlet peripheral edge.

### CROSS-REFERENCE TO RELATED APPLICATION

This application claims priority to U.S. provisional application Ser. No. 63/547,993, filed on Nov. 10, 2023.

### BACKGROUND OF THE INVENTION

### 1. Field of the Invention

The present invention relates to the field of implantable medical devices. More particularly, the present invention relates to an active implantable medical device (AMID) that is configured for placement in the skull and is adapted for remote charging and to communicate with a remote clinician programmer, and the like.

### 2. Prior Art

Implantable medical devices usually include a communication antenna for remote two-way communication and, for devices having a secondary power source, a separate charging antenna.

Implantable medical devices conventionally have a metallic housing that can be hermetically sealed to protect internal electronic components connected to the power source from body fluids. However, a metallic housing prevents positioning the charging and communication antennas in the housing. That is because the metal of the housing prevents both remote charging signals from reaching the power source and electromagnetic coupling between the communication antenna and external systems, such as a remote programmer or interrogation device. For that reason, the charging and communication antennas are typically placed in a molded polymeric header, for example, a molded epoxy header that is connected to the metallic housing through a feedthrough assembly.

For the specific application of an implantable neurostimulator where the active implantable medical device is placed on or in the skull, a craniectomy is first performed to provide a skull cavity. An open tray is placed in the skull cavity and the tray is screwed to the skull. The tray is typically made from metal so that screw tabs extending outwardly from the tray can be adjusted by bending them to follow the anatomy and geometry of the skull. The neurostimulator is then nested in the open tray with the neurostimulator facing away from the metal tray. However, even though the neurostimulator faces away from the metal tray, the metallic material of the tray behind the polymeric header supporting the charging and communication antennas can negatively affect remote charging and severely limit communication between the implanted device and a remote programmer or interrogation device. That is because the metal tray behind the molded header can cause eddy currents during charging and remote communication that can severely degrade charging efficiency and prevent proper remote communication.

It is also a trend in recent developments related to implantable medical devices to remove a section of a metal housing and replace it with a ceramic winder as an insert. The communication and charging antennas are then contained inside the modified device housing behind the ceramic window. While this improvement enables the device manufacturer to remove the communication and charging antenna from the molded header and put them inside the device housing, this solution is not entirely satisfactory. That is because having the ceramic window completely framed by the metal housing means that eddy currents can still develop during charging and remote communication.

In contrast, the ceramic insert of the present invention includes an upstanding sidewall that takes the place of a removed portion of the upstanding main sidewall of the main tray for an implantable medical tray. Providing a medical tray where the metal material of the tray does not completely frame the ceramic insert helps to reduce eddy currents during charging of the power source and during two-way remote communication.

Therefore, there is a need for a medical tray, for example, a craniometry tray, where a portion of the metallic tray including its sidewall has been replaced with a ceramic insert. With an active implantable medical device nested in the medical tray, the ceramic insert portion of the tray is aligned with and behind the molded header supporting the charging and communication antennas. Placing the ceramic portion of the tray behind the charging and communication antennas significantly enhances coupling efficiency of the antennas to external devices. Moreover, making the remainder of the medical tray from metal means that the tray maintains the functionality of bendable screw tabs for securing the tray to patients of various anatomies by allowing the tabs to be bent into the proper geometry during the implant procedure.

### SUMMARY OF THE INVENTION

The metallic housing of an implantable medical device can interfere with two-way communication between the medical device and an associated patient programmer or clinician programmer. These programmers are used by the patient and the clinician to configure the medical device to operate in a desired manner. A patient programmer is used by the patient in whom the medical device is implanted to adjust the parameters of electrical stimulation delivered by the device. A clinician programmer is used by medical personnel to configure and adjust stimulation parameters that the patient is not permitted to control. However, a metallic device housing may interfere with the inductive signals that are transmitted back and forth from the patient and clinician programmers to the medical device. A metallic device housing may also interfere with the inductive or RF charging signals that are used to recharge an electrical power source contained inside the medical device. To overcome these shortcomings, the communication/charging antenna, whether there are two dedicated antennas or an integrated antenna, must be placed outside the metallic housing, typically inside a polymeric header connected to the device housing.

However, when the implantable medical device is a neurostimulator that is intended for use in a craniometry, the medial device is nested in a metal medical tray that had previously been positioned in a skull cavity. Even though the metal medical tray is an open facing tray, the metal of the tray that is positioned behind the polymeric header supporting the charging and communication antennas can still cause eddy currents during charging and two-way communication that can severely degrade charging and communication efficiency.

A solution to this problem is to replace the portion of the metallic tray that is aligned behind the polymeric header with a ceramic insert. The insert has a conformal shape that is similar to or that matches the portion of the metal tray that has been removed. The ceramic insert is joined to the metallic tray by a braze. The joining process is done by first supporting a metallization on an edge of the ceramic insert. Next, a braze pre-form is positioned between the metallized edge of the ceramic insert and a conformal edge of the metallic tray. This ceramic insert/braze pre-form/metallic tray assembly is then subjected to a brazing process to melt the braze material so that when the braze cools, it has sealed to the metallized edge of the insert and to the conformal edge of the metallic tray. Gold is a preferred material for the braze pre-form.

These and other aspects of the present invention will become increasingly more apparent to those skilled in the art by reference to the following detailed description and to the appended drawings.

### BRIEF DESCRIPTION OF THE DRAWINGS

FIG. 1 is a simplified block diagram of an exemplary medical system 10 according to various embodiments of the present invention.

FIG. 2 is an exploded view of a neurostimulator 12 and one embodiment of an associated craniometry tray 30 comprising a main tray 32 and a ceramic insert 34 according to the present invention.

FIG. 3 is a perspective view of the neurostimulator 12 nested in the craniometry tray 30 illustrated in FIG. 2.

FIG. 4 is an exploded view of a neurostimulator 12 and another embodiment of an associated craniometry tray 130 comprising a main tray 132 and a ceramic insert 134 according to the present invention.

FIG. 5 is a perspective view of the neurostimulator 12 nested in the craniometry tray 130 illustrated in FIG. 4.

### DETAILED DESCRIPTION OF THE PREFERRED EMBODIMENTS

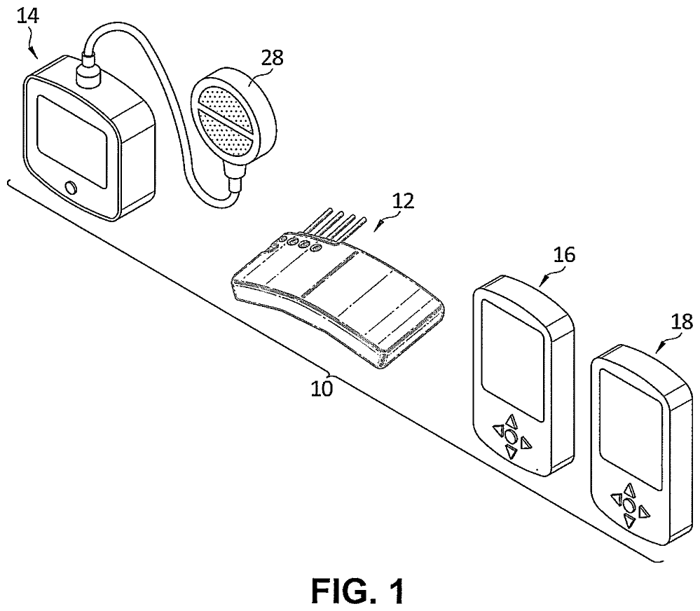

Turning now to the drawings, FIG. 1 illustrates a simplified block diagram of an exemplary medical system 10 according to the present invention. The medical system 10 includes an active implantable medical device (AIMD) 12, for example, a neurostimulator which is configured for implantation in a patient's body tissue. The medical system 10 also has an external charger 14, a patient programmer 16, and a clinician programmer 18.

The neurostimulator 12 comprises a hermetically sealed housing 20 made from a biocompatible and biostable metal, preferably titanium or stainless-steel, connected to a molded header 22. As is well known by those skilled in the art, the housing 20 contains a printed circuit board (PCB) assembly (not shown) connected to an electrical energy power source (not shown). The PCB supports at least one, and preferably a plurality of electronic components as an assembly that controls the various functions performed by the neurostimulator 12. These include, but are not limited to, receiving sensed electrical signals pertaining to functions of the body tissue in which the neurostimulator 12 is implanted and for delivering electrical current pulses to the body tissue through implantable leads 24 (FIGS. 1 to 4).

The molded header 22 connected to the device housing 20 is made from a polymeric material, for example, TECOTHANE©, which is a thermoplastic polyurethane marketed by Lubrizol Advanced Materials, Inc. for use in medical applications. The header 22 supports a plurality of terminal blocks (not shown) that are electrically connected to the electronic circuitry of the PCB contained inside the device housing 20 through a hermitic feedthrough assembly (not shown). The terminal blocks are each configured to detachably receive the proximal electrical contact of an implantable lead 24. Four leads 24 are shown extending from the implantable neurostimulator 12 illustrated in FIGS. 1 to 4, however, that is exemplary and the neurostimulator 12 can have more or less than four leads. The proximal electrical contact of each lead 24 is secured to its terminal block supported in the molded header 22 by a set screw 26. Each lead 24 has electrical conductors that extend from its electrical contact received in a terminal block to at least one and, preferably, at least two distal electrodes (not shown). The distal electrodes are secured to body tissue to provide electrical stimulation to the tissue and to sense biological signals from the body tissue.

In one embodiment, the molded header 22 also supports a communication antenna (not shown) and a charging coil (not shown) connected to a charging circuit supported on the PCB contained inside the device housing 20. The charging circuit is configured to convert RF or inductive energy signals received by the charging coil from an external charging pad 28 connected to the external charger 14 (FIG. 1) into a direct current voltage to charge the electrical power source to power the electronic components of the PCB. The communication antenna is configured for transmitting and receiving communication signals between the neurostimulator 12 and external medical devices. For more detail regarding a changing coil and a communication antenna supported in the molded header of an implantable medical device, reference is made to U.S. Pat. No. 8,929,986 to Parker et al., which is incorporated herein by reference.

In another embodiment of the present invention, a dual-function coil that is configured to receive both inductive charging and telemetry signals is supported in the molded header 22. U.S. Pat. No. 9,750,930 to Chen, which is assigned to the assignee of the present invention and incorporated herein by reference, describes such a coil.

The patient programmer 16 and the clinician programmer 18 may be portable handheld devices, such as a smartphone or other custom device, that are used to communicate with a communication antenna, or the dual-function coil described by the '930 patent and supported in the molded header 22 to configure the neurostimulator 12 so that the medical device can operate in a desired manner. The patient programmer 16 is used by the patient in whom the neurostimulator 12 is implanted. The patient may adjust the parameters of electrical stimulation delivered by the neurostimulator 12, such as by selecting a stimulation program, changing the amplitude and frequency of the electrical stimulation, among other parameters, and by turning stimulation on and off.

The clinician programmer 18 is used by medical personnel to configure the other system components and to adjust stimulation parameters that the patient is not permitted to control. These include setting up stimulation programs among which the patient may choose and setting upper and lower limits for the patient's adjustments of amplitude, frequency, and other parameters. It is also understood that although FIG. 1 illustrates the patient programmer 16 and the clinician programmer 18 as two separate devices, they may be integrated into a single programmer in some embodiments.

Whether or not the neurostimulator 12 contains an electrical power source, electrical power may be delivered to the neurostimulator 12 through the external charging pad 28 connected to the external charger 14. In some embodiments, the charging pad 28 is configured to directly power the neurostimulator 12 or it is configured to charge a rechargeable electrical energy power source contained inside the device housing 20. The external charging pad 28 can be a hand-held device that is connected to the external charger 14, or it can be an internal component of the external charger 14.

The electrical power source contained inside the device housing 20 can be a capacitor or a rechargeable battery, for example a hermetically sealed rechargeable Li-ion battery. However, the electrical energy power source is not limited to any one chemistry or even a rechargeable chemistry and can be of an alkaline cell, a primary lithium cell, a rechargeable lithium-ion cell, a Ni/cadmium cell, a Ni/metal hydride cell, a supercapacitor, a thin film solid-state cell, and the like. Preferably, the electrical energy power source is a lithium-ion electrochemical cell comprising a carbon-based or Li₄Ti₅O₁₂-based anode and a lithium metal oxide-based cathode, such as of LiCoO₂ or lithium nickel manganese cobalt oxide (LiNiₐMn_{b}Co_{1-a-b}O₂). The electrical energy power source can also be a solid-state thin film electrochemical cell having a lithium anode, a metal-oxide based cathode and a solid electrolyte, such as an electrolyte of LiPON (LiₓPO_{y}N_{z}).

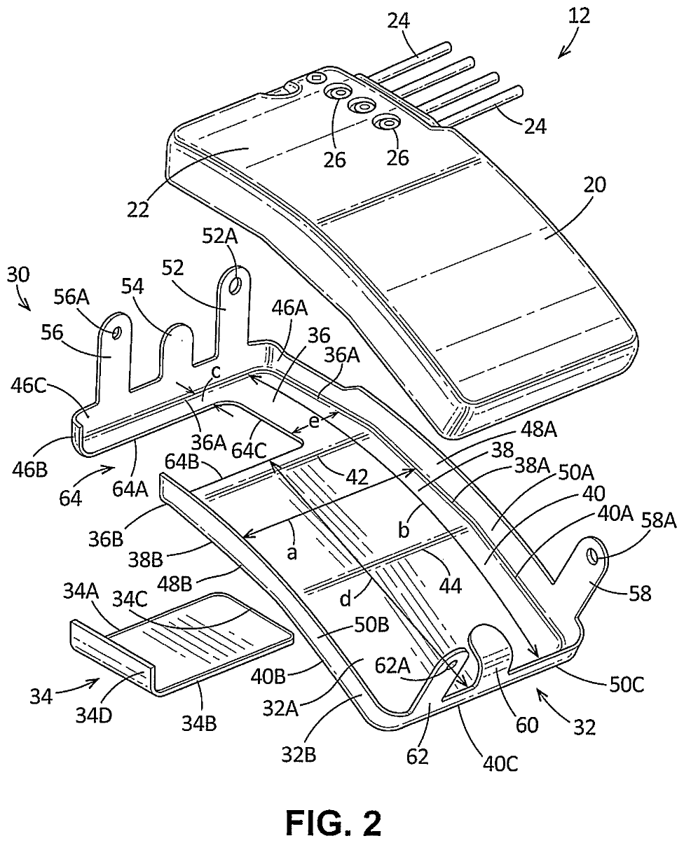

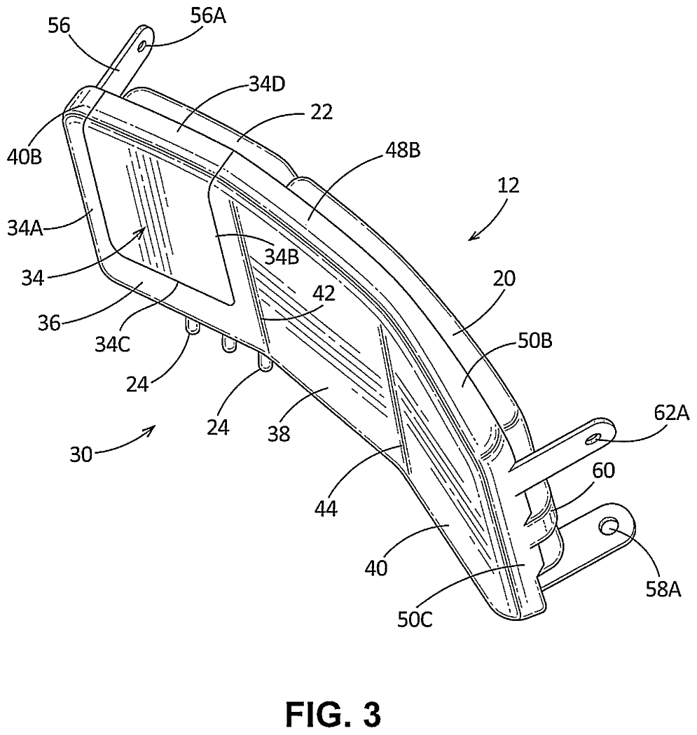

Referring now to FIGS. 2 and 3, the exemplary neurostimulator 12 is shown nested in one embodiment of a medical tray, for example, a craniometry tray 30 according to the present invention. The craniometry tray 30 includes a metallic main tray 32 and a ceramic insert 34. In general, the metallic main tray 32 is made from titanium or stainless-steel and comprises a base plate extending to and meeting with an upstanding main sidewall. An inlet extends inwardly through the main sidewall and into the base plate with the inlet having an inlet peripheral edge. The ceramic insert 34 comprises a planar plate portion that extends to an upstanding insert sidewall so that the insert has an insert peripheral with a conformal shape that matches that of the inlet in the main tray. As used herein, the term “conformal” means that the periphery of the ceramic insert has the same shape, outline, or contour as the shape of the periphery of the inlet into the main tray.

In greater detail, the main tray 32 comprises a base plate 32A that extends to and is continuous with an upstanding main sidewall 32B. The base plate 32A has three generally planar plate portions comprising a proximal plate portion 36, an intermediate plate portion 38 and a distal plate portion 40. The proximal and intermediate plate portions 36 and 38 meet at a proximal bend 42 so that the plate portions 36, 38 are offset with respect to each other at an obtuse angle ranging from about 160° to about 175°.

Similarly, the intermediate and distal plate portions 38 and 40 meet at a distal bend 44 so that the plate portions 38, 40 are offset with respect to each other at an obtuse angle ranging from about 160° to about 175°. That way, the proximal and distal plate portions 36 and 40 are offset with respect to each other at an angle ranging from about 10° to about 40° with the intermediate plate 38 connected to them at the respective proximal and distal bends 42 and 44. This angled relationship between the proximal, intermediate, and distal plate portions 36, 38 and 40 provides a contoured shape to the main tray 32 that is designed to both follow the contour of the neurostimulator 12 and of the patient's skull to enhance comfort with the neurostimulator nested in the craniometry tray 30 positioned in a craniometry cavity (not shown) formed in the skull.

The proximal plate portion 36 is a planar plate that is comprised of spaced-apart right and left sides 36A and 36B that extend to an intermediate proximal end 36C opposite the proximal bend 42. The right side 36A meets the proximal end 36C at a rounded right corner. Similarly, the left side 36B meets the proximal end 36C at a rounded left corner.

The intermediate plate portion 38 is a planar plate that is comprised of spaced-apart right and left sides 38A and 38B that extend to the proximal and distal bends 42, 44.

The distal plate portion 40 is a planar plate that is comprised of spaced-apart right and left sides 40A and 40B that extend to an intermediate distal end 40C opposite the distal bend 44. The right side 40A meets the distal end 40C at a rounded right corner and the left side 40B meets the distal end 40C at a rounded left corner.

Further, the upstanding main sidewall 32B has a proximal right main sidewall segment 46A that extends upwardly from the proximal right side 36A, a proximal left main sidewall segment 46B that extends upwardly from the proximal left side 36B, and a proximal main sidewall segment 46C that extends upwardly from the proximal end 36C of the proximal plate portion 36.

The upstanding main sidewall 32B further comprises an intermediate right main sidewall segment 48A that extends upwardly from the intermediate right side 38A, and an intermediate left main sidewall segment 48B that extends upwardly from the intermediate left side 38B of the intermediate plate portion 38.

The upstanding main sidewall 32B also has a distal right main sidewall segment 50A that extends upwardly from the distal right side 40A, a distal left main sidewall segment 50B that extends upwardly from the distal left side 40B, and a distal main sidewall segment 50C that extends upwardly from the distal end 40C of the distal plate portion 40.

FIGS. 2 and 3 also illustrate that a number of spaced-apart and bendable primary metal tabs are integral with and extend upwardly from the upstanding main sidewall 32B. In particular, primary tabs 52, 54 and 56 extend upwardly from the proximal main sidewall segment 46C of the proximal plate portion 36, primary tab 58 extends upwardly from the distal right main sidewall segment 50A of the distal plate portion 50. Secondary metal tabs 60 and 62 are integral with and extend upwardly from the distal main sidewall segment 50C of the distal plate portion 40. The secondary tabs 54 and 60 are somewhat shorter than the primary tabs 52, 56, 58 and 62. With the neurostimulator 12 nested in the craniometry tray 30, the shorter secondary tabs 54 and 60 are bent inwardly to secure the medical device in position. Since the secondary tabs 54 and 60 serve to capture the neurostimulator 12 in the medical tray 30, they do not have openings. The somewhat longer primary tabs 52, 56, 58 and 62 are configured to be bent outwardly. A fastener, for example, a bone screw (not shown), is received in their respective openings 52A, 56A, 58A and 62A to secure the medical tray 30 containing the neurostimulator 12 in place in a skull cavity. The openings 52A, 56A, 58A and 62A are spaced from the main tray 32 a sufficient distance so that a bone screw moved through the opening and threaded into bone, for example, the skull is spaced from the main tray.

As particularly shown in FIG. 2, the craniometry tray 30 has a width labeled “a” spanning from the right main sidewall segments 46A, 48A and 50A to the left main sidewall segments 46B, 48B and 50B, and a length labeled “b” spanning from the proximal main sidewall segment 46C to the distal main sidewall segment 50C.

Further, an inlet 64 extends inwardly into the proximal left main sidewall segment 46B and the proximal left side 36B of the proximal plate portion 36. The inlet 64 has an inlet peripheral edge that is bounded by opposed proximal and distal edges 64A and 64B that extend to a lateral edge 64C. The proximal edge 64A is spaced from the proximal end 36C of the proximal plate portion 36 by a distance labeled “c”, the distal edge 64B is spaced from the distal end 40C of the distal plate portion 40 by a distance labeled “d”, and the lateral edge 64C is spaced from the proximal right side 36A of the proximal plate portion 36 by a distance labeled “e”. The distance labeled “c”, “d” and “e” can vary depending on a particular design for the medical tray 30, however, they cannot be zero. This means that there must be some plate material between the proximal edge 64A of the inlet 64 and the proximal end 36C of the proximal plate portion 36 and between the lateral edge 64C and the right proximal side 36A of the proximal plate portion 36.

In an alternate embodiment according to the present invention, the inlet 64 extends inwardly into the proximal right main sidewall segment 46A and the proximal right side 36A of the proximal plate portion 36. As with the embodiment of the inlet 64 illustrated in FIGS. 2 and 3, in this alternate embodiment for the inlet, there must be some plate material between the proximal edge 64A of the inlet 64 and the proximal end 36C of the proximal plate portion 36 and between the lateral edge and the left proximal side 36B of the proximal plate portion 36.

The insert 34 of the craniometry tray 30 is formed from a ceramic material that has a relatively high dielectric constant. An essentially pure alumina is a preferred ceramic material. The term “essentially pure alumina” refers to an alumina ceramic having the chemical formula Al₂O₃. “Essentially pure” means that the post-sintered alumina is at least 96% alumina. In a preferred embodiment, the post-sintered alumina is at least 99% high purity alumina. Other suitable ceramic materials include zirconium oxide, and boron nitride.

Prior to sintering, the alumina comprising the insert 34 may be a paste, a slurry, or a green-state ceramic that is injection molded, powder pressed, and the like, into the desired shape as a single monolithic structure. The green-state alumina ceramic is very pliable due to organic binders and solvents that have been temporarily added to the system.

After being formed into the desired shape for the insert 34, the green-state ceramic is subjected to a controlled sintering process in ambient air that comprises a binder bakeout portion, a sintering portion, and a cool down portion. The binder bakeout portion is performed at a temperature that ranges from about 400° C. to about 700° C. for a minimum of about 4 hours. A preferred binder bakeout is at a temperature that ranges from about 550° C. to about 650° C. A more preferred binder bakeout is at a temperature that ranges from about 500° C. to about 600° C.

The sintering portion of the controlled process for the preferred alumina insert 34 is preferably performed at a temperature that ranges from about 1,400° C. to about 1,900° C. for up to about 6 hours. A preferred sintering profile has a temperature that ranges from about 1,500° C. to about 1,800° C. A more preferred sintering temperature ranges from about 1,600° C. to about 1,700° C.

The cool down portion occurs either by turning off the heating chamber and allowing the chamber to equalize to room temperature or, preferably by setting the cool down portion at a rate of up to about 5° C./min from the hold temperature cooled down to about 1,000° C. At about 1,000° C., the chamber is allowed to naturally equalize to room temperature. A more preferred cool down is at a rate of about 1° C./min from the hold temperature to about 1,000° C. and then the heating chamber is allowed to naturally equalize to room temperature. Once the binders and solvents have been driven out of the ceramic and sintering has occurred, the result is a solid monolithic high purity alumina insert 34 comprising a planar plate portion bounded by opposed proximal and distal edges 34A and 34B extending to a lateral edge 34C. A sidewall 34D extends upwardly from the planar plate opposite the lateral edge 34C.

To connect the insert 34 to the main tray 32 of the craniometry tray 30, a metallization (not shown) is supported on the insert peripheral edge comprising the opposed proximal and distal edges 34A and 34B, on the lateral edge 34C of the planar plate portion and on the opposed edges of the sidewall 34D extending upwardly from the planar plate portion of the ceramic insert 34. A suitable metallization comprises two metallization layers, a first adhesion layer that is directly applied to the outwardly facing edge of the insert 34, and a second, wetting layer, which is applied on top of the adhesion layer. In a preferred embodiment, the adhesion layer is titanium, and the wetting layer is either molybdenum or niobium.

The adhesion and wetting metallization layers may be applied to the ceramic insert 34 by thin and thick film technologies, such as printing, painting, plating, and deposition processes. Metallization processes include screen printing, pad printing, brush coating, direct bonding, active metal brazing, magnetron sputtering, physical vapor deposition, ion implantation, electroplating, and electroless plating. In an alternate embodiment, both the adhesion and wetting metallization layers may be provided by a single metallization layer. It is noted that in the present drawings, the adhesion and wetting layers are intentionally not shown for the sake of simplicity.

A braze pre-form, for example a gold preform (not shown), is seated on the metallized peripheral edges 34A, 34B and 34C including the proximal and distal edge of the sidewall 34D. The main tray 32/gold pre-form/ceramic insert 34 subassembly is then subjected to a brazing process, as is well known to those skilled in the art related to brazing a ceramic material to a metallic workpiece. The brazing process melts the gold to thereby join the main tray 32 to the ceramic insert 34.

While at least two leads 24 are needed for a functioning device, it is also within the scope of the present invention that the neurostimulator 12 can have more or less than the four leads 24 illustrated in FIGS. 2 to 4. The leads 24 are powered by the previously described electrical power source, or they can be powered through RF or inductive energy transmitted from the external charging pad 28 connected to the external charger 14 to the charging coil electrically connected to the PCB contained inside the device housing 20. The PCB supports electronic components that control the various functions of the medical device, including having electrodes at the distal end of the leads 24 deliver electrical stimulation to a patient and sense biological signals from body tissue.

However, when it is desired to recharge the electrical power source, RF or inductive energy is transmitted from the external charging pad 28 connected to the external charger 14 to the charging coil disposed in the molded header 22. An important aspect of the present invention is that the charging coil in the header 22 is aligned with the ceramic insert 34. Even though the craniometry tray is an open structure, if the tray were made entirely from metal, for example, titanium, the titanium on the back side of the header would create eddy currents that could interfere with charging efficiency. In some cases, the charging efficiency could be degraded which means that more time is needed to recharge the power source. In a worst case, charging efficiency could be degraded to an extent that charging the power source is not possible. However, the ceramic insert 34 of the present invention readily permits the transmission of RF or inductive energy from the external charging pad 28 connected to the external charger 14 to the charging coil disposed in the molded header 22 with substantially no degradation of charging efficiency due to eddy currents. This is a significant improvement attributed to the craniometry tray 30 of the present invention.

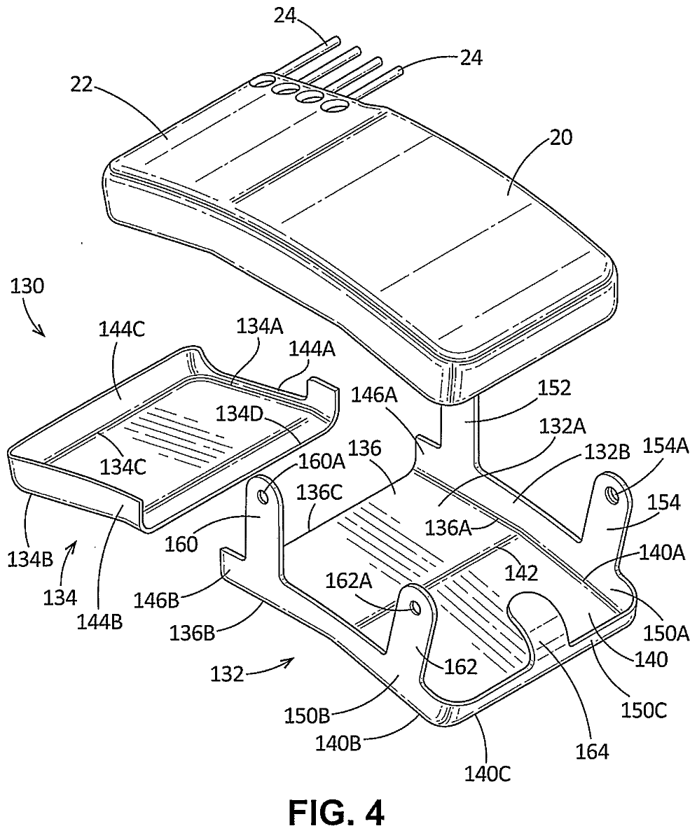

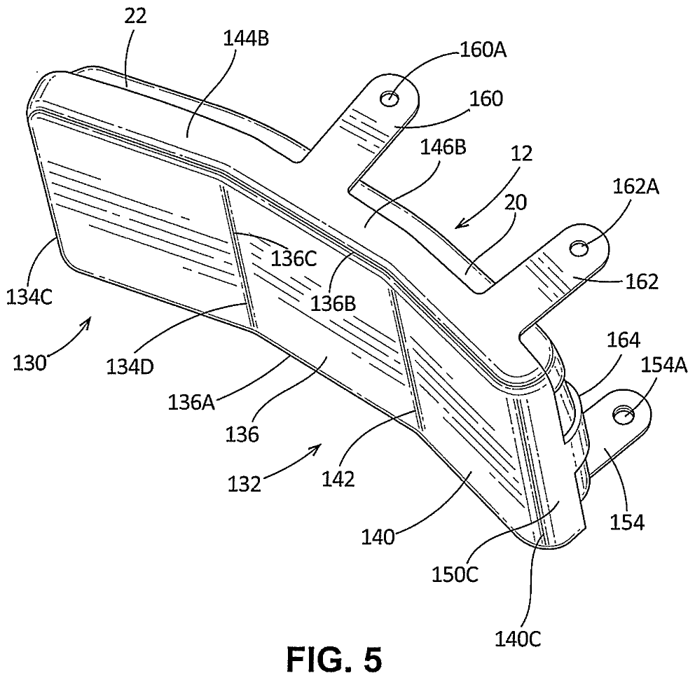

Referring now to FIGS. 4 and 5, the exemplary neurostimulator 12 is shown nested in another embodiment of a medical tray, for example, a craniometry tray 130 according to the present invention. The craniometry tray 130 includes a main tray 132 and a ceramic insert 134. The main tray 132 is made from titanium or stainless-steel and comprises a base plate 132A that extends to and is continuous with an upstanding main sidewall 132B. The base plate 132A has two planar plate portions comprising a proximal plate portion 136 and a distal plate portion 140. The proximal and distal plate portions 136 and 140 meet at a bend 142 so that the plate portions 136, 140 are offset with respect to each other at an obtuse angle ranging from about 160° to about 175°. This angled relationship between the proximal and distal plate portions 136 and 140 provides a contoured shape to the main tray 132 that is designed to both follow the contour of the neurostimulator 12 and of the patient's skull to enhance comfort with the neurostimulator nested in the craniometry tray 130 positioned in a craniometry cavity (not shown) formed in the skull.

The proximal plate portion 136 is a planar plate that is comprised of spaced-apart right and left sides 136A and 136B that extend to an intermediate proximal edge 136C opposite the bend 142.

The distal plate portion 140 is a planar plate that is comprised of spaced-apart right and left sides 140A and 140B that extend to a distal end 140C opposite the bend 142. The right side 140A meets the distal end 140C at a rounded right corner and the left side 140B meets the distal end 140C at a rounded left corner.

Further, the upstanding main sidewall 132B has a proximal right main sidewall segment 146A that extends upwardly from the proximal right side 136A, and a proximal left main sidewall segment 1463 that extends upwardly from the proximal left side 136B. The proximal right main sidewall segment 146A, the intermediate proximal edge 136C and the proximal left main sidewall segment 146B collectively extend to a proximal peripheral edge of the proximal plate portion 136.

The upstanding main sidewall 132B also has a distal right main sidewall segment 150A that extends upwardly from the distal right side 140A, a distal left main sidewall segment 150B that extends upwardly from the distal left side 140B, and a distal main sidewall segment 150C that extends upwardly from the distal end 140C of the distal plate portion 140.

FIGS. 4 and 5 also illustrate that a number of spaced-apart bendable primary metal tabs extend upwardly from the upstanding main sidewall 132B. In particular, primary tabs 152 and 154 extend upwardly from the respective proximal and distal main sidewall segments 146A and 150A of the proximal and distal plate portions 136 and 140, and primary tabs 160 and 162 extend upwardly from the respective proximal and distal left main sidewall segments 146B and 150B of the proximal and distal plate portions 136 and 140.

A somewhat shorter secondary tab 164 extends upwardly from the distal main sidewall segment 150C of the distal plate portion 140. With the neurostimulator 12 nested in the craniometry tray 130, the shorter secondary tab 164 is bent inwardly to secure the medical device in position. Since the secondary tab 164 serves to capture the neurostimulator 12 in the medical tray 130, it does not have an opening. However, the somewhat longer primary tabs 152, 154, 160 and 162 are configured to be bent outwardly. A fastener, for example, a bone screw (not shown), is received in their respective openings (opening for tab 152 is not shown), 154A, 160A and 162A to secure the medical tray 130 containing the neurostimulator 12 in place in a skull cavity. The openings 154A, 160A and 162A are spaced from the main tray 132 a sufficient distance so that a bone screw moved through the opening and threaded into bone, for example, the skull is spaced from the main tray.

The insert 134 of the craniometry tray 130 is formed from a ceramic material, preferably alumina, in a similar process as previously described for the insert 34 illustrated in FIGS. 2 and 3. The insert 134 has a planar plate portion bounded by opposed right and left sides 134A and 134B extending to a proximal end 134C opposite a distal lateral edge 134D. A right sidewall segment 144A extends upwardly from the right side 134A, a left sidewall segment 144B extends upwardly from the left side 134B and an intermediate sidewall segment 144C extends upwardly from the proximal end 134C of the planar plate portion of the ceramic insert 134. The right and left sidewall segments 144A and 144B and the distal lateral edge 134D collectively extend to a distal insert peripheral edge of the ceramic insert 134. The distal edge of the ceramic insert 134 has a shape that matches the shape of the proximal edge of the proximal plate portion 136 of the main tray 132.

To connect the ceramic insert 134 to the main tray 132 to form the craniometry tray 130, the distal edge formed by the exposed ends of the right and left sidewall segments 144A and 144B and the distal lateral edge 134D of the ceramic insert 134 are provided with a metallization (not shown) in a similar manner as previously described with respect to the ceramic insert 34 shown in FIGS. 2 and 3. A braze pre-form, for example a gold preform, (not shown) is then seated on the metallized peripheral edge comprising the distal edges of the right and left sidewall segments 144A and 144B and the distal lateral edge 134D. The main tray 132/gold pre-form/ceramic insert 134 subassembly is then subjected to a brazing process, as is well known to those skilled in the art related to brazing a ceramic material to a metallic workpiece. The brazing process melts the gold to thereby join the main tray 132 to the ceramic insert 134 to thereby form the craniometry tray 130.

Then, with the neurostimulator 12 nested in the craniometry tray 130, and when it is desired to recharge the electrical power source of the medical device, RF or inductive energy is transmitted from the external charging pad 28 connected to the external charger 14 to the charging coil disposed in the molded header 22 of the neurostimulator. As previously described with respect to the medical tray 30 illustrated in FIGS. 2 and 3, an important aspect of the present invention is that the charging coil in the header 22 is aligned with the ceramic insert 134 portion of the medical tray 130. Even though the craniometry tray 130 is an open structure, if the tray were made entirely from metal, for example, titanium, the titanium on the back side of the header could create eddy currents that could interfere with charging efficiency. In some cases, the charging efficiency could be degraded which means that more time is needed to recharge the power source. In a worst case, charging efficiency could be degraded to an extent that charging the power source would not be possible. However, the ceramic insert 34 of the present invention readily permits the transmission of RF or inductive energy from the external charging pad 28 connected to the external charger 14 to the charging coil disposed in the molded header 22 with substantially no degradation of charging efficiency due to eddy currents. This is a significant improvement attributed to the craniometry tray 130 of the present invention.

In that respect, the ceramic inserts 34 and 134 comprising the craniometry trays 30 and 130 of the present invention do not interfere with the communication/charging fields that are transmitted between external devices and the medical device 12 when the neurostimulator 12 is nested in the craniometry tray 30, 130 and the tray is implanted in body tissue, for example, the skull of a patient. These include communication signals from patient and clinician programmers to the neurostimulator 12, and inductive or RF (radio frequency) charging signals sent from an external charging transmitter to the charging antenna supported in the molded header 22 and connected to the power source for the medical device.

It is appreciated that various modifications to the inventive concepts described herein may be apparent to those skilled in the art without departing from the spirit and scope of the present invention as defined by the hereinafter appended claims.

1. A medical tray, comprising:
a) a metallic main tray comprising a base plate extending to and meeting with an upstanding main sidewall, wherein an inlet extends inwardly through the main sidewall and into the base plate with the inlet having an inlet peripheral edge;
b) a ceramic insert comprising a planar plate portion extending to and meeting with an upstanding insert sidewall, wherein the insert has an insert peripheral edge having an insert shape that is conformal with an inlet shape of the inlet peripheral edge;
c) a metallization supported on the insert peripheral edge; and
d) a braze joining the metallization supported on the insert peripheral edge to the inlet peripheral edge.

2. The medical tray of claim 1, wherein the inlet peripheral edge in the main tray comprises a base plate edge portion meeting an upstanding main sidewall edge portion.

3. The medical tray of claim 1, wherein the insert peripheral edge comprises a planar plate edge portion meeting an upstanding insert sidewall edge portion.

4. The medical tray of claim 1, wherein the base plate of the main tray has a base plate length extending from a proximal end to a distal end and a base plate width extending from a right side to a left side of the base plate, and wherein the inlet extends inwardly through the upstanding main sidewall extending upwardly from one of the right side and the left side of the base plate, but the inlet does not extend to the other of the right and left side of the base plate.

5. The medical tray of claim 4, wherein the inlet extends inwardly through the upstanding main sidewall extending upwardly from one of the right side and the left side of the base plate, but the inlet does not extend to either the proximal end or the distal end of the base plate.

6. The medical tray of claim 4, wherein the inlet extending inwardly through the upstanding main sidewall extending upwardly from one of the right side and the left side of the base plate is spaced closer to the proximal end than to the distal end of the base plate.

7. The medical tray of claim 1, wherein the metallization comprises a titanium adhesion layer that is directly applied to the insert peripheral edge and a wetting layer of either molybdenum or niobium that is applied on top of the titanium adhesion layer.

8. The medical tray of claim 1, wherein the braze joining the metallization supported on the insert peripheral edge to the inlet peripheral edge is gold.

9. The medical tray of claim 1, wherein the main tray is comprised of titanium.

10. The medical tray of claim 1, wherein the ceramic insert is selected from alumina, zirconium oxide, and boron nitride.

11. The medical tray of claim 1, wherein the main tray further comprises at least two spaced-apart upstanding and bendable primary tabs, each provided with an opening.

12. The medical tray of claim 1, wherein the main tray further comprises at least two spaced-apart upstanding and bendable secondary tabs that are not provided with an opening.

13. The medical tray of claim 1, wherein the base plate and the upstanding main sidewall of the metallic main tray have a contoured shape that is designed to follow a contour of a patient's skull.

14. A medical tray, comprising:
a) a metallic main tray comprising a base plate extending to and meeting with an upstanding main sidewall, wherein an inlet extends inwardly through the main sidewall and into the base plate so that the inlet has an inlet peripheral edge including opposed edge portions of the upstanding main sidewall;
b) a ceramic insert comprising a planar plate portion bounded by opposed proximal and distal insert edges extending to a lateral insert edge opposite an upstanding insert sidewall;
c) a metallization supported on the opposed proximal and distal insert edges, on the lateral insert edge and on opposed edges of the upstanding insert sidewall; and
d) a braze joining the metallization supported on the ceramic insert to the metallic main tray.

15. The medical tray of claim 14, wherein the base plate of the main tray has a base plate length extending from a proximal end to a distal end and a base plate width extending from a right side to a left side of the base plate, and wherein the inlet extends inwardly through the upstanding main sidewall extending upwardly from one of the right side and the left side of the base plate, but the inlet does not extend to the other of the right and left side of the base plate.

16. The medical tray of claim 14, wherein the inlet extends inwardly through the upstanding main sidewall extending upwardly from one of the right side and the left side of the base plate, but the inlet does not extend to either the proximal end or the distal end of the base plate.

17. The medical tray of claim 14, wherein the inlet extending inwardly through the upstanding main sidewall extending upwardly from one of the right side and the left side of the base plate is spaced closer to the proximal end than to the distal end of the base plate.

18. The medical tray of claim 14, wherein the metallization comprises a titanium adhesion layer that is directly applied to the insert peripheral edge and a wetting layer of either molybdenum or niobium that is applied on top of the titanium adhesion layer.

19. The medical tray of claim 14, wherein the main tray further comprises at least two spaced-apart upstanding and bendable primary tabs, each provided with an opening, and at least two spaced-apart upstanding and bendable secondary tabs that are not provided with an opening.

20. A medical tray, comprising:
a) a metallic main tray comprising a base plate extending to and meeting with an upstanding main sidewall, wherein the base plate including the upstanding main sidewall extend to a proximal edge;
b) a ceramic insert comprising an insert plate portion bounded by opposed right and left insert sidewalls and an intermediate sidewall segment forming a distal edge of the ceramic insert;
c) a metallization supported on the distal edge of the ceramic insert; and
d) a braze joining the metallization supported on the distal edge of the ceramic insert to the proximal edge of the metallic main tray.
